(19) United States
(12) Patent Application Publication
(10) Pub. No.: US 2025/0204855 A1
(43) Pub. Date: Jun. 26, 2025

(54) DEVICE AND SYSTEM FOR MEASURING HEAD AND FACIAL WEARING PRESSURE

(71) Applicant: GOERTEK TECHNOLOGY CO., LTD., Qingdao (CN)

(72) Inventors: Zhiwei HU, Qingdao (CN); Luan ZHANG, Qingdao (CN); Shizhan SUN, Qingdao (CN)

(21) Appl. No.: 19/074,824

(22) Filed: Mar. 10, 2025

Related U.S. Application Data
(63) Continuation of application No. PCT/CN2022/127751, filed on Oct. 26, 2022.

(30) Foreign Application Priority Data
Oct. 8, 2022 (CN) 202211231536.5

(51) Int. Cl. A61B 5/00; A42B 1/019

(52) U.S. Cl. CPC A61B 5/6843; A42B 1/019

(57) ABSTRACT

The present application relates to the technical field of wearable devices, and specifically discloses a device and system for measuring head and facial wearing pressure, including a human head model and a flexible pressure sensor. The human head model includes a hard bottom shell and a flexible skin layer provided outside the hard bottom shell. The flexible pressure sensor is provided outside the detection area of the flexible skin layer. The flexible pressure sensor includes an insulating pad and a plurality of conductive threads, the plurality of conductive threads are interlacingly woven on the insulating pad, and two conductive threads at horizontal and vertical intersections are separated by the insulating pad to serve as pressure detection points.

### CROSS-REFERENCE TO RELATED APPLICATIONS

This application is a continuation application of International Application No. PCT/CN2022/127751, filed on Oct. 26, 2022, which claims priority to Chinese Patent Application No. 202211231536.5, filed on Oct. 8, 2022. The disclosures of the above-mentioned applications are incorporated herein by reference in their entireties.

### TECHNICAL FIELD

The present application relates to the technical field of wearable devices, and in particular to a device and a system for measuring head and facial wearing pressure.

### BACKGROUND

Head and facial wearing pressure measurement, as an objective description of wearing comfort evaluation, plays a vital role in wearing comfort evaluation. It can describe wearing comfort from a more objective perspective, quantify the differences between solutions, and guide the modification of design solutions.

Most of the existing flexible pressure measurement solutions use polyethylene terephthalate (PET) film printed carbon-based silver paste circuits as sensing points, which cannot meet the softness, accuracy, and fit requirements for large curved facial surfaces. In addition, the shapes of existing pressure sensors are mostly regular, which cannot meet the customization requirements with high degrees of freedom.

In summary, how to solve the problem that existing pressure sensors are difficult to effectively measure the head and facial wearing pressure is urgent for those skilled in the art to solve.

### SUMMARY

The main objective of the present application is to provide a device and system for measuring head and facial wearing pressure, the structural design of which can effectively solve the problem that existing pressure sensors are difficult to effectively measure the head and facial wearing pressure.

In order to achieve the above objective, the following technical solutions.

A device for measuring head and facial wearing pressure, including: a human head model including a hard bottom shell and a flexible skin layer provided outside the hard bottom shell; and a flexible pressure sensor provided outside a detection area of the flexible skin layer.

In an embodiment, the flexible pressure sensor includes an insulating pad and a plurality of conductive threads, the plurality of conductive threads are interlacingly woven on the insulating pad, and two conductive threads at horizontal and vertical intersections are separated by the insulating pad to serve as pressure detection points.

In an embodiment, a density of the pressure detection points ranges from 2 to 10 points per cm².

In an embodiment, a thickness of the flexible pressure sensor ranges from 0.5 mm to 3 mm.

In an embodiment, a diameter of the conductive thread ranges from 0.2 mm to 0.8 mm.

In an embodiment, the flexible skin layer includes a silicone layer or a foam layer.

In an embodiment, the detection area includes a frontal plane area, an overhead area, an around-ear area, and a back of head area, and the flexible pressure sensor includes: a frontal plane part corresponding to the frontal plane area; an overhead part corresponding to the overhead area; an around-ear part corresponding to the around-ear area; and a back of head part corresponding to the back of the head area, the frontal plane part is provided with a through hole avoiding eyes, and the around-ear part is provided with a through hole avoiding the ears.

In an embodiment, the conductive threads are blended threads including a conductive metal thread and a fiber thread.

In an embodiment, the fiber thread is a super-hydrophobic fiber thread, and the blended threads include a plurality of super-hydrophobic fiber threads respectively provided around the conductive metal thread.

In an embodiment, the super-hydrophobic fiber threads are nanofiber threads.

The device for measuring head and facial wearing pressure provided by the present application includes a human head model and a flexible pressure sensor. The human head model includes a hard bottom shell and a flexible skin layer, and the flexible skin layer is provided outside the hard bottom shell. The flexible pressure sensor is provided outside the detection area of the flexible skin layer, and the flexible pressure sensor includes an insulating pad and a plurality of conductive threads. The plurality of conductive threads are woven in a horizontal and vertical manner on the insulating pad, and the two conductive threads at the horizontal and vertical intersections are separated by the insulating pad to serve as pressure detection points.

In the present application, the human head model can simulate the head and facial. During the test, the head and facial wearable device is stably worn on the device for measuring head and facial wearing pressure, and the flexible pressure sensor on the surface of the human head model contacts and is squeezed with the wearable device. The squeezing force in the contact area will cause the pressure detection point formed by the flexible pressure sensor (i.e., the horizontal and vertical intersection of the conductive thread) to output a changing electrical signal. The change in the electrical signal is collected by the matching collector, and the corresponding pressure test result can be obtained after processing by the processor. In summary, the device for measuring head and facial wearing pressure provided by the present application has a flexible pressure sensor with good flexibility, and it has a good fit with the human head model, thereby meeting the pressure detection requirements of the head and facial wearable device. In addition, the flexible pressure sensor is in direct contact with the head and facial wearable device, so that a more accurate pressure distribution boundary can be obtained, avoiding the pressure dispersion problem caused by indirect measurement, and improving the pressure measurement accuracy.

In order to achieve the above-mentioned objective, the present application further provides a system for measuring head and facial wearing pressure, which includes any of the above-mentioned device for measuring head and facial wearing pressures. Since the above-mentioned device for measuring head and facial wearing pressure has the above-mentioned technical effects, the system for measuring head and facial wearing pressure having the device for measuring head and facial wearing pressure should also have corresponding technical effects.

### BRIEF DESCRIPTION OF THE DRAWINGS

In order to more clearly illustrate the technical solutions in the embodiments of the present application or in the related art, drawings used in the embodiments or in the related art will be briefly described below. Obviously, the drawings in the following description are only some embodiments of the present application. It will be apparent to those skilled in the art that other figures can be obtained based on the structures shown in the drawings without creative work.

FIG. 1 is a schematic front view of a device for measuring head and facial wearing pressure according to an embodiment of the present application.

FIG. 2 is a schematic side view of FIG. 1.

FIG. 3 is a schematic cross-sectional view of the device for measuring head and facial wearing pressure of FIG. 1.

FIG. 4 is a schematic diagram of a partial cross-sectional structure of the flexible pressure sensor in FIG. 3.

FIG. 5 is a schematic diagram of the structure of the conductive thread in FIG. 4.

FIG. 6 is a schematic diagram of a system for measuring head and facial wearing pressure according to an embodiment of the present application.

FIG. 7 is a pressure cloud diagram obtained by measurement.

### DETAILED DESCRIPTION OF THE EMBODIMENTS

Embodiments of the present application disclose a device and system for measuring head and facial wearing pressure to effectively measure the head and facial wearing pressure.

The following will be combined with the drawings in the embodiments of the present application to clearly and completely describe the technical solutions in the embodiments of the present application. Obviously, the described embodiments are only part of the embodiments of the present application, not all of the embodiments. Based on the embodiments in the present application, all other embodiments obtained by those skilled in the art without creative work are within the scope of protection of the present application.

The present application provides a device and system for measuring head and facial wearing pressure that can be used for wearing pressure testing of head and facial wearable devices. Specific head and facial wearable devices include but are not limited to headphones, virtual reality (VR) glasses, augmented reality (AR) glasses, mixed reality glasses (MR) and other devices.

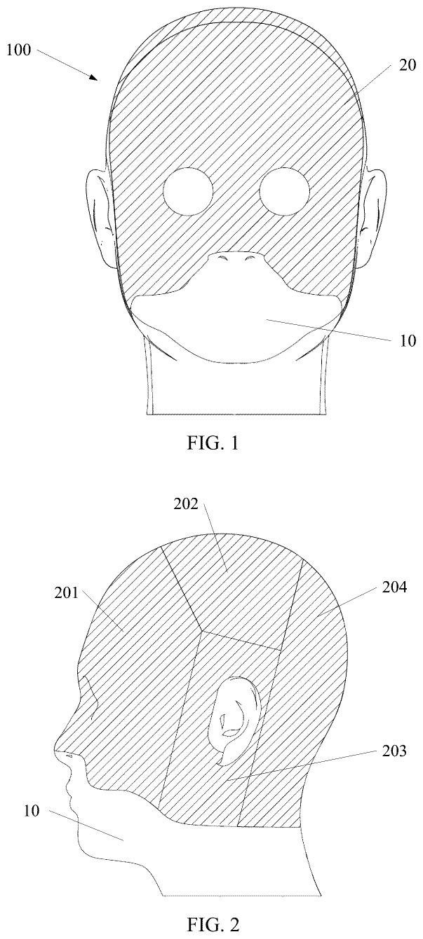

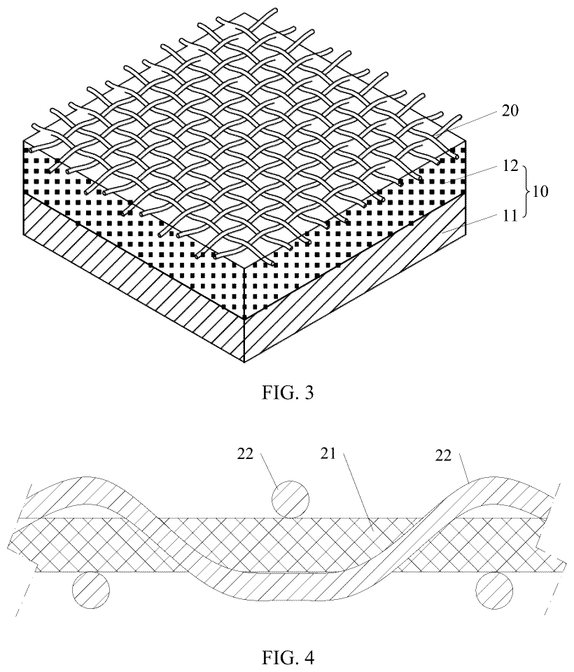

As shown in FIG. 1 to FIG. 4, FIG. 1 is a front view structural schematic diagram of a device for measuring head and facial wearing pressure according to an embodiment of the present application. FIG. 2 is a side view schematic diagram of FIG. 1. FIG. 3 is a cross-sectional structural schematic diagram of the device for measuring head and facial wearing pressure of FIG. 1. FIG. 4 is a partial cross-sectional structural schematic diagram of the flexible pressure sensor in FIG. 3.

In an embodiment, the device 100 for measuring head and facial wearing pressure provided by the present application includes a human head model 10 and a flexible pressure sensor 20. The human head model 10 includes a hard bottom shell 11 and a flexible skin layer 12. The hard bottom shell 11 is used to simulate bone support, and the flexible skin layer 12 is provided outside the hard bottom shell 11 and used to simulate skin, which can be deformed under pressure. The specific shape of the human head model 10 can be obtained through big data analysis. The flexible pressure sensor 20 is provided outside the detection area of the flexible skin layer 12, and the specific position of the detection area can be set according to the wearing position and wearing manner of the head and facial wearable device. The flexible pressure sensor 20 includes an insulating pad 21 and a plurality of conductive threads 22. The plurality of conductive threads 22 are woven horizontally and vertically on the insulating pad 21, and the two conductive threads 22 at the horizontal and vertical intersections are separated by the insulating pad 21 as pressure detection points. The insulating pad 21 is used as the substrate, which has good flexibility and can fit well with the human head model 10. The conductive thread 22 is woven on the insulating pad 21, that is, the insulating pad 21 can support and position the conductive thread 22. The two conductive threads 22 at the horizontal and vertical intersections are separated by the insulating pad 21. When the head and facial wearable device is stably worn on the human head model 10, the distance between the two wires will change due to the squeezing effect of the head and facial wearable device, so that a changing electrical signal can be output, such as a changing resistance value between the two wires. That is, the horizontal and vertical intersection can be used as the detection point of the flexible pressure sensor 20, and the pressure situation at the detection point can be reflected by collecting the changing electrical signal of the detection point.

By applying the device 100 for measuring the head and facial wearing pressure provided by the present application, the human head model 10 can simulate the head and facial part. During the test, the head and facial wearable device is stably worn on the device 100 for measuring the head and facial wearing pressure, and the flexible pressure sensor 20 on the surface of the human head model 10 contacts and is squeezed with the wearable device. The squeezing force in the contact area will cause the pressure detection point formed by the flexible pressure sensor 20, that is, the horizontal and vertical intersection of the conductive thread 22, to output a changing electrical signal. The change in the electrical signal is collected by the matching collector 200, and the corresponding pressure test result can be obtained after processing by the processor. In summary, in the device 100 for measuring the head and facial wearing pressure provided by the present application, the flexible pressure sensor 20 has good flexibility, and it has a good fit with the human head model 10, thereby meeting the pressure detection requirements of the head and facial wearable device.

In addition, the flexible pressure sensor 20 is in direct contact with the head and facial wearable device. Compared with the solution with built-in pressure sensor, such as the pressure sensor is provided between the soft rubber layer and the support layer, it is not in direct contact with the head and facial wearable device to be measured, but the soft rubber layer is compressed by the head and facial wearable device to be measured, and the soft rubber layer transmits the pressure to the pressure sensor. Since the pressure is not completely transmitted vertically in the direction of the pressure when it is transmitted in the soft rubber layer, but it will diffuse laterally, the pressure image and pressure value collected by the pressure sensor will have deviations ranging from 20% to 65%. And the degree of diffusion is affected by the thickness of the soft rubber layer and the pressure area, and it cannot be effectively predicted and eliminated by software algorithms later. In the present application, the flexible pressure sensor 20 is in direct contact with the head and facial wearable device to be measured, so that a more accurate pressure distribution boundary can be obtained, avoiding the pressure dispersion problem caused by indirect measurement, and improving the pressure measurement accuracy.

Furthermore, the conductive threads 22 are woven into the insulating pad 21. In an embodiment, the conductive threads 22 can be directly woven together with the insulating pad 21, thereby avoiding the increase in thickness caused by secondary packaging.

In an embodiment, the density of the pressure detection points ranges from 2 to 10 points/cm², that is, the quantity of horizontal and vertical intersections of the conductive thread 22 per square centimeter is 2 to 10. The specific density of the pressure detection points can be 2 points/cm², 3 points/cm², 4 points/cm², 5 points/cm², 6 points/cm², 7 points/cm², 8 points/cm², 9 points/cm², 10 points/cm², etc. The density of the pressure detection points is controlled within the above range, which can take into account both the detection accuracy and the processing calculation amount. In other embodiments, the density of the pressure detection points can also be adjusted according to the detection needs.

In an embodiment, the thickness of the flexible pressure sensor 20 ranges from 0.5 mm to 3 mm. In an embodiment, the thickness of the flexible pressure sensor 20 is 0.5 mm, 0.8 mm, 1 mm, 1.3 mm, 1.5 mm, 1.8 mm, 2 mm, 2.3 mm, 2.5 mm, 2.7 mm, 3 mm, etc. In combination with the thread diameter of the conductive thread 22 and the thickness of the insulating pad 21, the thickness of the flexible pressure sensor 20 is controlled to be 0.5 mm to 3 mm, which can obtain a good detection effect. In addition, according to the range design of the flexible pressure sensor 20, the thickness of the insulating pad 21 can be set accordingly. In other embodiments, the thickness of the flexible pressure sensor 20 can also be adjusted according to the detection needs.

In an embodiment, the diameter of the conductive thread 22 ranges from 0.2 mm to 0.8 mm. In an embodiment, the diameter of the conductive thread 22 is 0.2 mm, 0.3 mm, 0.4 mm, 0.5 mm, 0.6 mm, 0.7 mm, 0.8 mm, etc. The diameter of the conductive thread 22 within the above numerical range can take into account the softness and plastic deformation ability of the flexible pressure sensor 20, so that it can be stably attached to the surface of the human head model 10 to accurately reflect the wearing pressure. In other embodiments, the diameter of the conductive thread 22 can also be adjusted according to the detection needs.

In an embodiment, the flexible skin layer 12 includes a silicone layer or a foam layer. The use of a silicone layer or a foam layer can realistically simulate the skin state, so that when the head and facial wearable device is stably worn on the human head model 10, the flexible skin layer 12 can realistically simulate the skin compression and deformation state. In other embodiments, the flexible skin layer 12 can also be made of other soft and deformable materials.

In an embodiment, the thickness of the flexible skin layer 12 may range from 1 mm to 16 mm. In an embodiment, the thickness of the flexible skin layer 12 is 1 mm, 3 mm, 5 mm, 8 mm, 10 mm, 13 mm, 16 mm, etc. When the thickness of the flexible skin layer 12 is within the above numerical range, the real state of the skin can be better simulated.

In an embodiment, the hard bottom shell 11 is made of polycarbonate (PC) or nylon. The above materials have high hardness and are not easily deformed, so they can well simulate the state of bone support, and have low cost and light weight. In other embodiments, the hard bottom shell 11 can also be made of other hard and non-deformable materials such as plastics.

In an embodiment, as shown in FIG. 2, the detection area includes the frontal plane area, the overhead area, the around-ear area and the back of the head area. The flexible pressure sensor 20 includes a frontal plane part 201 corresponding to the frontal plane area, an overhead part 202 corresponding to the overhead area, an around-ear part 203 corresponding to the around-ear area, and a back of the head part 204 corresponding to the back of the head area. The frontal plane part 201 has a through hole that avoids the eyes, and the around-ear part 203 has a through hole that avoids the ears. The frontal plane part 201, the overhead part 202, the around-ear part 203 and the back of the head part 204 can be connected as a whole, that is, an integrated flexible pressure sensor 20 can be used, or it can be a split structure that is separately set. The above detection area can cover 90% of head shapes. In other embodiments, the detection area can also be set according to the wearing position of the head and facial wearable device, and is not limited to the above settings.

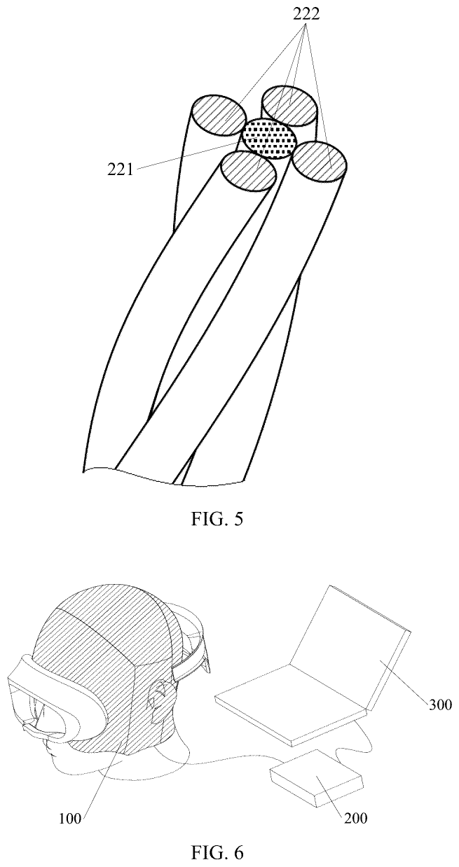

In an embodiment, as shown in FIG. 5, the conductive thread 22 is a blended wire including a conductive metal thread 221 and a fiber thread 222. In order to meet the conductive properties of the conductive fiber, the conductive metal thread 221 is mixed into the fiber thread 222, and the wire diameter of the conductive metal thread 221 is adjusted so that the flexible pressure sensor 20 can obtain plastic deformation ability under the premise of meeting a certain degree of softness, so that it is easier to stably attach to the measurement area with a large curvature change, thereby improving the accuracy of pressure measurement.

In an embodiment, the fiber thread 222 is a super-hydrophobic fiber thread, and the blended threads includes a plurality of super-hydrophobic fiber threads, which are respectively provided around the conductive metal thread 221. The conductive metal thread 221 is surrounded by a plurality of super-hydrophobic fiber threads, and the waterproof property of the super-hydrophobic fiber threads is utilized to make the guide thread have a waterproof function.

In an embodiment, the super-hydrophobic fiber thread is a nanofiber thread. The super-hydrophobic property of the nanofiber thread is utilized to make the conductive thread 22 waterproof, and the nanofiber thread preparation process is simple. In other embodiments, the super-hydrophobic fiber thread can also be obtained by forming a super-hydrophobic layer on the surface of the fiber thread 222.

Based on the device 100 for measuring head and facial wearing pressure provided in the above embodiment, the present application further provides a system for measuring head and facial wearing pressure. As shown in FIG. 6, the system for measuring head and facial wearing pressure includes a collector 200, a collection processor 300 and any device 100 for measuring head and facial wearing pressure in the above embodiment, and the collector 200 is electrically connected to the collection processor 300. Since the system for measuring head and facial wearing pressure adopts the device 100 for measuring head and facial wearing pressure in the above embodiment, the beneficial effects of the system for measuring head and facial wearing pressure are shown in the above embodiments.

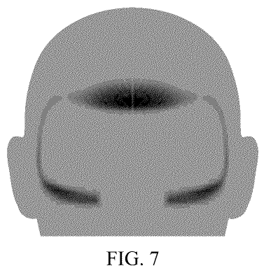

During the test, the head and facial wearable device is stably worn on the device 100 for measuring head and facial wearing pressure, and the flexible pressure sensor 20 attached to the surface of the human head model 10 contacts and is squeezed with the head and facial wearable device. The squeezing force in the contact area causes the flexible pressure sensor 20 to output an electrical signal. The collector 200 collects the changes in the electrical signal and transmits the collected electrical signal to the collection processor 300 through a data line or wirelessly, such as a computer. After the processing of the built-in matching software of the collection processor 300, a pressure image as shown in FIG. 7 can be obtained, thereby intuitively displaying the pressure distribution and size. For the specific collection manner and principle of the collector 200 can be referred to the related art, which will not be repeated here. The pressure image output by the collection processor 300 after software processing can be specifically presented in the form of a pressure cloud map, with different colors or grayscales to represent the pressure size. The software can output the real-time pressure value currently measured, and can record and save the pressure data of the test process. In other embodiments, the collection processor 300 can also process the received electrical signal to obtain the pressure value of each pressure detection point, and display it in a chart or other manner.

In an embodiment, the device 100 for measuring head and facial wearing pressure can obtain different pressure value ranges by calibrating the flexible pressure sensor 20 to meet the wearing pressure measurement requirements of different products. The sampling frequency of the collector 200 is adjustable from 1 Hz to 1000 Hz, and can collect pressure signals in motion.

The various embodiments in this specification are described in a progressive manner, and each embodiment focuses on the differences from other embodiments. The same or similar parts between the various embodiments can be referenced to each other.

The above description of the disclosed embodiments enables those skilled in the art to implement or use the present application. Various modifications to these embodiments will be apparent to those skilled in the art, and the general principles defined herein may be applied to other embodiments without departing from the spirit or scope of the present application. Accordingly, the present application is not to be limited to the embodiments shown herein but is to be accorded the broadest scope consistent with the principles and novel features disclosed herein.

What is claimed is:

1. A device for measuring head and facial wearing pressure, comprising:
a human head model comprising a hard bottom shell and a flexible skin layer provided outside the hard bottom shell; and
a flexible pressure sensor provided outside a detection area of the flexible skin layer; wherein the flexible pressure sensor comprises an insulating pad and a plurality of conductive threads; the plurality of conductive threads are interlacingly woven on the insulating pad, and two conductive threads at horizontal and vertical intersections are separated by the insulating pad to serve as pressure detection points.

2. The device for measuring head and facial wearing pressure of claim 1, wherein a density of the pressure detection points ranges from 2 to 10 points per cm².

3. The device for measuring head and facial wearing pressure of claim 1, wherein a thickness of the flexible pressure sensor ranges from 0.5 mm to 3 mm.

4. The device for measuring head and facial wearing pressure of claim 1, wherein a diameter of the conductive thread ranges from 0.2 mm to 0.8 mm.

5. The device for measuring head and facial wearing pressure of claim 1, wherein the flexible skin layer comprises a silicone layer or a foam layer.

6. The device for measuring head and facial wearing pressure of claim 1, wherein the detection area comprises a frontal plane area, an overhead area, an around-ear area, and a back of head area, and the flexible pressure sensor comprises:
a frontal plane part corresponding to the frontal plane area;
an overhead part corresponding to the overhead area;
an around-ear part corresponding to the around-ear area; and
a back of head part corresponding to the back of the head area;
wherein the frontal plane part is provided with a through hole avoiding eyes, and the around-ear part is provided with a through hole avoiding the ears.

7. The device for measuring head and facial wearing pressure of claim 1, wherein the conductive threads are blended threads comprising a conductive metal thread and a fiber thread.

8. The device for measuring head and facial wearing pressure of claim 7, wherein the fiber thread is a super-hydrophobic fiber thread, and the blended threads comprise a plurality of super-hydrophobic fiber threads respectively provided around the conductive metal thread.

9. The device for measuring head and facial wearing pressure of claim 8, wherein the super-hydrophobic fiber threads are nanofiber threads.

10. A system for measuring head and facial wearing pressure, comprising:
a collector;
a collection processor electrically connected to the collector; and
the device for measuring head and facial wearing pressure of claim 1;
wherein the device for measuring head and facial wearing pressure is electrically connected to the collector.
